(19) United States
(12) Patent Application Publication
(10) Pub. No.: US 2018/0235900 A1
(43) Pub. Date: Aug. 23, 2018

(54) SUBCUTANEOUS RESERVOIR DEVICE AND METHOD OF MANUFACTURE

(71) Applicant: Research Triangle Institute, Research Triangle Park, NC (US)

(72) Inventors: Stephanie Lynn Swarner, Cary, NC (US); Leah Johnson, Durham, NC (US); Robert F. Praino, JR., Westwood, MA (US); Phillip Gregory Durham, Broadway, NC (US); Mark E. Kleiman, Canton, MA (US); Ginger Denison Rothrock, Cary, NC (US); Ariane van der Straten Ponthoz, Kensington, CA (US)

(21) Appl. No.: 15/890,171

(22) Filed: Feb. 6, 2018

Related U.S. Application Data
(60) Provisional application No. 62/454,978, filed on Feb. 6, 2017.

(51) Int. Cl. A61K 9/70; A61K 9/00; A61K 47/34; A61K 31/685; C07K 16/10; B29C 43/18; B29C 43/56; B29C 65/08; B29C 65/00

(52) U.S. Cl. CPC A61K 9/7007; A61K 9/0019; A61K 47/34; A61K 31/685; C07K 16/1045; B29C 43/18; B29K 2067/00; B29C 65/08; B29C 66/723; B29C 66/727; B29C 66/731; B29C 66/7352; B29C 2043/561; B29C 43/56

(57) ABSTRACT

The medical devices of the present disclosure are filled reservoirs such as a cylinder comprised of a polymer film which contains a reservoir of active agent, plus excipient, in some cases, for disease prevention, treatment, and/or contraception. The polymer film is permeable to the active agent after subcutaneous implantation of the device into a body. The cylinder is comprised by the lamination of one or two polymer films which are ultrasonically welded to contain the drug material. The use of an ultrasonic welding process enables sealing of the polymer films to create the closed cylinder. The medical device is useful for long term disease prevention, such as prevention of HIV infection.

### CROSS-REFERENCE TO RELATED APPLICATION

This application claims the benefit of priority of U.S. Provisional Patent Application No. 62/454,978 titled “SUBCUTANEOUS RESERVOIR DEVICE AND METHOD OF MANUFACTURE” and filed on Feb. 6, 2017, which is incorporated herein in its entirety by this reference.

### FEDERAL FUNDING LEGEND

The invention was made with Government support under Federal Grant No. AID-OAA-A-14-00012 awarded by the United States Agency for International Development. The Government has certain rights in the invention.

### TECHNICAL FIELD

The presently disclosed subject matter relates to a subcutaneous reservoir device for delivery of an active agent over an extended period of time and method of manufacture thereof.

### BACKGROUND

Women's need for effective biomedical interventions for HIV prevention and contraception remains worldwide. Despite several landmark successes, Pre-Exposure Prophylaxis (PrEP) trials and demonstration projects have persistently struggled with suboptimal adherence to pre-/peri-coital and daily administration of oral or vaginal products. Sustained, user-independent delivery of PrEP alleviates users from burdensome time- or event-driven regimens and bypasses many adherence challenges of user-dependent methods. Also, systemic administration combined with long-term delivery may significantly protect a wider variety of HIV exposure routes, including vaginal, rectal, and parenteral. Currently, two nanosuspension injectable agents are under investigation for PrEP: TMC278LA (an NNRTI) and S/GSK1265744 (an integrase inhibitor). However, key drawbacks exist: (a) in the event of a drug-related serious adverse event, the product cannot be removed; and (b) the first-order dissolution of the nanosuspended drug results in a first-order pharmacokinetic (PK) profile with a gradual decrease, resulting in a potentially long “tail” extending beyond the effective duration of the dose in which suboptimal levels of drug continue to be delivered systemically.

Similarly, there is an unmet need for a long-acting biodegradable implant for a contraceptive method. Although long-acting contraceptives are preferable for longer duration of protection and greater effectiveness, a quick return to fertility is believed to be important from an end-user perspective.

While there remains an urgent need for effective oral, vaginal, and rectal HIV PrEP agents, trials and demonstration projects have been plagued with poor adherence to oral and topically administered pre/peri-coital and daily product regimens (Van Damme, Corneli et al. 2012, Marrazzo, Ramjee et al. 2015). This is of particular concern in Sub-Saharan Africa, where HIV incidence remains highest globally and adherence to daily product use has been unexpectedly low. This was exemplified most recently by the undesirable FemPrEP and VOICE trial results, where adherence to a daily product was particularly low among young women, who continue to be at highest risk of HIV infection (Marrazzo, Ramjee et al. 2015). Recent results from two Phase III clinical trials for PrEP-based Vaginal rings (VRs) (i.e., The Ring Study and ASPIRE) showed promising results for preventing HIV in women, but also suggested low adherence, particularly in women under 21 via post ad hoc analysis (Baeten, Palanee-Phillips et al., Nel 2016). Low adherence inhibits the ability to determine a product's biological efficacy or safety, and thus complicates interpretation of trial results, but will also importantly undermine the effectiveness of products brought to market. Currently, two nanosuspension injectable agents for long-acting PrEP are under investigation: TMC278LA (an NNRTI) and S/GSK1265744 (an integrase inhibitor) (Abraham and Gulick 2012). In both cases the formulation is delivered intra-muscularly as a nanosuspension. Intravenous and subcutaneous infusion of bNAbs has shown great promise for PrEP in Phase I studies (Caskey, Klein et al. 2015, Ledgerwood, Coates et al. 2015). The antibody-mediated prevention (AMP) trial is an on-going Phase II evaluation of VRC01 for prevention of HIV. These clinical studies have revealed several limitations. Specifically, large intravenous doses may be necessary for protection against HIV-1 in humans; thus, more work to improve the potency and increase the plasma half-life is critical for allowing less frequent dosing at smaller volumes. Furthermore, although subcutaneous (SC) dosing has clear advantages over intravenous administration for PrEP, SC administration has been limited by the poor tolerability of high injection volumes by patients. Finally, different variants of antiretroviral (ARV) may have to be administered together to achieve an adequate breadth of coverage for prevention of HIV-1 (Ledgerwood, Coates et al. 2015). These limitations imposed by established antibody delivery routes are well-recognized, placing major restrictions on the facility and even efficacy of pharmacologic breakthroughs (Grainger 2004). Lastly, in the event of a drug related serious adverse event, the infused preventative cannot be removed.

Accordingly, there remains an unmet need for improved medical devices for treatment, prevention and contraception. The present disclosure provides such improved devices and methods of manufacture.

### SUMMARY OF THE DISCLOSURE

In some embodiments, the presently disclosed subject matter is directed to a reservoir device comprising an active agent contained within a reservoir. The reservoir is defined by one or more porous polymer membranes sealed with an ultrasonic weld, the porous membrane allowing for diffusion of the active agent through the pores of the membrane when positioned subcutaneously in a body of a subject.

In some embodiments, the presently disclosed subject matter is directed to a method for manufacturing a reservoir device for delivery of an active agent to a subject. Particularly, the method comprises imparting a vacuum to a first porous membrane positioned on a mold defining at least one cavity, wherein the first porous membrane takes a shape of the cavity in the presence of the vacuum. The method further comprises depositing an active agent into a portion of the first porous membrane that is received in the cavity, and positioning a second porous membrane carried on a release liner over the active agent and in contact with the first porous membrane. The method further comprises applying an ultrasonic force to a release liner positioned over the porous membrane(s) in an area surrounding the active agent to create a welded seal to contain the active agent within the cavity; and releasing the welded porous membranes from the mold and the release liner to provide a reservoir device(s). The porous membranes allow for diffusion of the active agent through the pores of the membrane when the reservoir device is positioned subcutaneously in a body of a subject.

In some embodiments, the presently disclosed subject matter is directed to a method for manufacturing a reservoir device for delivery of an active agent to a subject, the method comprising folding a porous membrane over to define a tubular cavity, depositing an active agent into the tubular cavity, and applying an ultrasonic force to the porous membrane to create a welded seal that contains the active agent within the tubular cavity providing a reservoir device. The porous membrane allows for diffusion of the active agent through the pores of the membrane when the reservoir device is positioned subcutaneously in a body of a subject.

In some embodiments, the presently disclosed subject matter is directed to a method for sustained delivery of an active agent to a subject, the method comprising implanting the disclosed reservoir device subcutaneously in a body of a subject, wherein diffusion of the active agent through the pores of the membrane of the device provides sustained delivery of the active agent to the subject for one or a combination of prevention, treatment, or contraception.

### BRIEF DESCRIPTION OF THE DRAWINGS

The foregoing aspects and other features of the disclosure are explained in the following description, taken in connection with the accompanying drawings, wherein:

FIG. 1 is an illustration of the general mechanism of a reservoir device in accordance with one or more embodiments of the presently disclosed subject matter.

FIG. 2A is a photograph of the disclosed thin film polymer reservoir device made from 80 kDa MW polycaprolactone film according to one or more embodiments of the presently disclosed subject matter.

FIG. 2B is a photograph of a marketed trochar (IMPLANON) showing utilization for implantation of the disclosed device according to one or more embodiments of the presently disclosed subject matter.

FIG. 3 is a line graph showing cumulative release (mg) of Tenofovir Alafenamide Fumarate (TAF), a nucleotide reverse transcriptase inhibitor (NRTI), from several Thin Film Polymer Devices (TFPDs) over time. Each line represents a single device according to one or more embodiments of the presently disclosed subject matter.

FIG. 4 is a line graph showing the daily release (mg/day) of Tenofovir Alafenamide Fumarate (TAF) from several Thin Film Polymer Devices (TFPDs) over time according to one or more embodiments of the presently disclosed subject matter.

FIG. 5A is a graph of plasma Tenofovir Alafenamide Fumarate (TAF) and tenofovir (TFV) concentrations over the course of 30 days from an individual New Zealand White rabbit after subcutaneous implantation with a Thin Film Polymer Device (TFPD) containing a high dose target of TAF of 1.5 mg/day according to one or more embodiments of the presently disclosed subject matter.

FIG. 5B is a graph of plasma Tenofovir Alafenamide Fumarate (TAF) and tenofovir (TFV) concentrations over the course of 30 days from an individual New Zealand White rabbit after subcutaneous implantation with a Thin Film Polymer Device (TFPD) containing a high dose target of TAF of 1.5 mg/day according to one or more embodiments of the presently disclosed subject matter.

FIG. 5C is a graph of plasma Tenofovir Alafenamide Fumarate (TAF) and tenofovir (TFV) concentrations over the course of 30 days from an individual New Zealand White rabbit after subcutaneous implantation with a Thin Film Polymer Device (TFPD) containing a high dose target of TAF of 1.5 mg/day according to one or more embodiments of the presently disclosed subject matter.

FIG. 5D is a graph of plasma Tenofovir Alafenamide Fumarate (TAF) and tenofovir (TFV) concentrations over the course of 30 days from an individual New Zealand White rabbit after subcutaneous implantation with a Thin Film Polymer Device (TFPD) containing a high dose target of TAF of 1.5 mg/day according to one or more embodiments of the presently disclosed subject matter.

FIG. 5E is a graph of plasma Tenofovir Alafenamide Fumarate (TAF) and tenofovir (TFV) concentrations over the course of 30 days from an individual New Zealand White rabbit after subcutaneous implantation with a Thin Film Polymer Device (TFPD) containing a high dose target of TAF of 1.5 mg/day according to one or more embodiments of the presently disclosed subject matter.

FIG. 5F is a graph of plasma Tenofovir Alafenamide Fumarate (TAF) and tenofovir (TFV) concentrations over the course of 30 days from an individual New Zealand White rabbit after subcutaneous implantation with a Thin Film Polymer Device (TFPD) containing a high dose target of TAF of 1.5 mg/day according to one or more embodiments of the presently disclosed subject matter.

FIG. 6A is a graph illustrating the tenofovir (TFV) concentration (nM) versus time (post implantation) for a high Tenofovir Alafenamide Fumarate (TAF) dose group (1.1 mg/day) and a middle TAF dose group (0.45 mg/day) according to one or more embodiments of the presently disclosed subject matter.

FIG. 6B illustrates images of one embodiment of a reservoir device prior to implementation (day 0) and post implementation (day 14) according to one or more embodiments of the presently disclosed subject matter.

FIG. 7 is a graph illustrating the cumulative mass of Tenofovir Alafenamide Fumarate (TAF) released (mg) versus time for reservoir devices of 18 kDa blend films (top and middle lines) or 80 kDa blend film (bottom line) according to one or more embodiments of the presently disclosed subject matter.

FIG. 8 is a graph illustrating the cumulative release of Tenofovir Alafenamide Fumarate (TAF) (mg) from Thin Film Polymer Devices (TFPD) having differing excipients over time according to one or more embodiments of the presently disclosed subject matter.

FIG. 9 is an illustration of a setup for manufacturing a reservoir device in accordance with one or more embodiments of the presently disclosed subject matter.

FIG. 10 is a photograph of a reservoir device in accordance with one or more embodiments of the presently disclosed subject matter.

FIG. 11 is an illustration of a layout for manufacturing a reservoir device in accordance with one or more embodiments of the presently disclosed subject matter.

FIG. 12A is an illustration of a layout of a cavity-shaping die for manufacturing a reservoir device in accordance with one or more embodiments of the presently disclosed subject matter.

FIG. 12B is an illustration of a cavity-shaping die in accordance with one or more embodiments of the presently disclosed subject matter.

FIG. 13A is a photograph illustrating a system for manufacturing a reservoir device in accordance with one or more embodiments of the presently disclosed subject matter.

FIG. 13B is a photograph illustrating a system for manufacturing a reservoir device in accordance with one or more embodiments of the presently disclosed subject matter.

FIG. 13C is a photograph illustrating a system for manufacturing a reservoir device in accordance with one or more embodiments of the presently disclosed subject matter.

FIG. 14 is an illustration of a setup for manufacturing a reservoir device in accordance with one or more embodiments of the presently disclosed subject matter.

### DETAILED DESCRIPTION OF THE DISCLOSURE

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

Articles “a” and “an” are used herein to refer to one or to more than one (i.e. at least one) of the grammatical object of the article. By way of example, “a reservoir device” means at least one reservoir device and can include more than one reservoir device.

Unless otherwise defined, all technical terms used herein have the same meaning as commonly understood by one of ordinary skill in the art to which this disclosure belongs.

The invention is a medical device and the associated process by which it can be produced for long acting delivery of an active agent such as an active pharmaceutical ingredient (API). The devices of the present disclosure address key drawbacks of existing technologies such as: (1) in the event of a drug-related serious adverse event, existing products and technologies cannot be removed; and (2) the first-order dissolution of nanosuspended drug in existing products and technologies results in a first-order pharmacokinetic (PK) profile with a gradual decrease, resulting in a potentially long “tail” extending beyond the effective duration of the dose in which suboptimal levels of drug continue to be delivered systemically. In contrast, the reservoir devices of the present invention provide zero-order drug release kinetics, resulting in a flat PK profile at a steady state. Upon depletion of drug from the implant, only a minimal tail can be expected according to the drug's half-life and supported by results described herein in the Examples. An example of one advantage of the disclosed devices is systemic administration, combined with long-term delivery, to significantly protect a wider variety of HIV exposure routes, including vaginal, rectal, and parenteral. This technology can be useful for a wide variety of therapeutics and preventatives, including small molecules and biologics.

The devices of the present invention also address the unmet need for a long-acting biodegradable implant for a contraceptive method. Although long-acting contraceptives are preferable for longer duration of protection and greater effectiveness, a quick return to fertility is believed to be important from an end-user perspective.

In one embodiment, the devices of the present invention are fabricated from the FDA-approved biodegradable polymer polycaprolactone (PCL) and provide a zero-order drug release profile to meet both of these needs. Finally, by combining both pregnancy and HIV prevention, the device offers women an empowering tool to protect themselves against multiple risks. Mulitpurpose Prevention Technologies (MPTs) that are simple, acceptable, and accessible hold great potential for significant impacts in public health. Women can receive dual protection discreetly, even if their stated intention is to address just one health need, because of pressures from their sociocultural context (e.g., HIV stigma) or relationships.

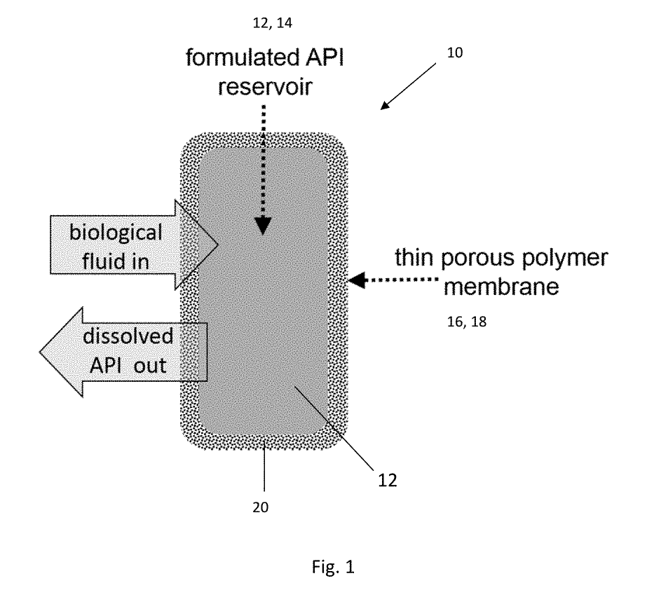

As shown in FIG. 1, device 10 comprises active agent 12 contained within reservoir 14 defined by one or more porous polymer membranes 16, 18 sealed with ultrasonic weld 20. Membranes 16, 18 are permeable to the active agent after implantation of the device into a body of a subject. In some embodiments, device 10 further comprises an excipient contained within reservoir 14. For example, the excipient can comprise PEG. Reservoir 14 can have a cylindrical shape, such as a cylinder with dimensions of 40×2.5 mm. In some embodiments, the cylinder can be formed by the lamination of one or both porous membranes 16, 18 that are ultrasonically welded to contain the active agent. The use of an ultrasonic welding process enables sealing of the membranes to create the closed cylinder. In one embodiment, device 10 can be used for long term disease prevention, such as prevention of HIV infection.

Active agent 12 can be one or a combination of a therapeutic, a preventative, or a contraceptive. In some embodiments, active agent 12 comprises an antibody, a small molecule, a protein, and/or a peptide. For example, in some embodiments, the active agent comprises an antibody for the prevention of HIV infection. In some embodiments, the active agent comprises a nucleotide reverse transcriptase inhibitor (NRTI) for treatment of HIV infection. The nucleotide reverse transcriptase inhibitor can be Tenofovir Alafenamide Fumarate (TAF).

Porous membranes 16, 18 allow for diffusion of the active agent through the pores of the membranes when positioned subcutaneously in a body of a subject. In some embodiments, membranes 16, 18 comprise polycaprolactone (PCL), poly(lactic-co-glycolic acid)(PLGA), or polylactic acid (PLA). For example, membranes 16, 18 can comprise polycaprolactone (PCL) at a molecular weight ranging from 15,000 to 80,000 kDa. In some embodiments, membranes 16, 18 have a thickness ranging from 1-30 μm or from 10-25 μm. The pore size of the disclosed membranes can range from 1-2 times the diameter of active agent 12. In some embodiments, porous polymer membranes 16, 18 are biodegradable.

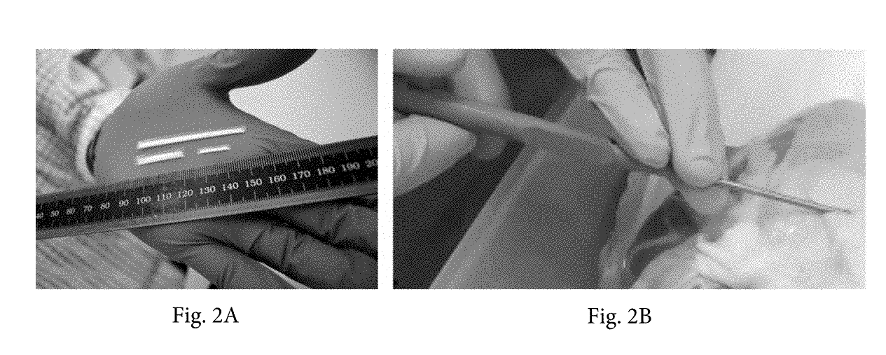

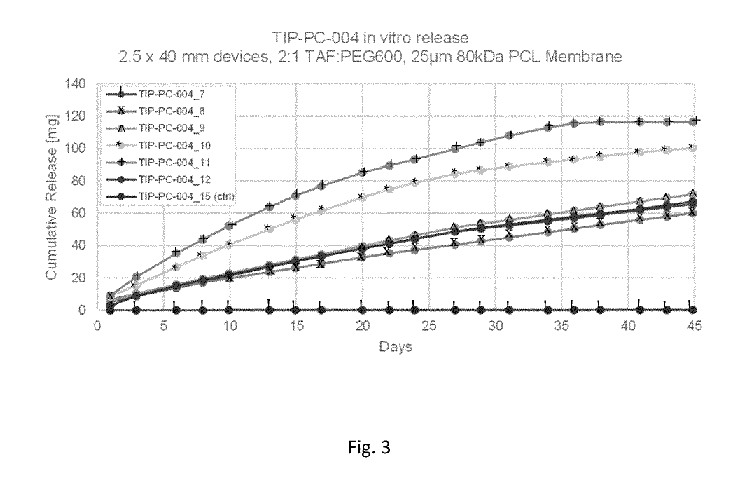

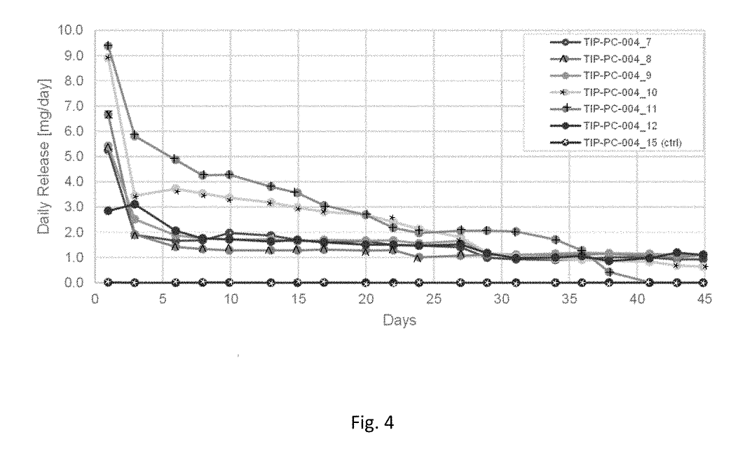

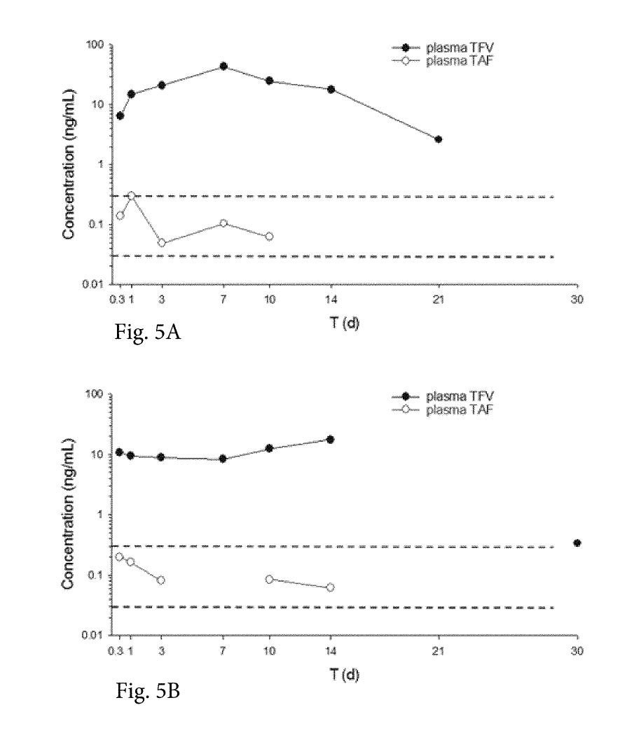

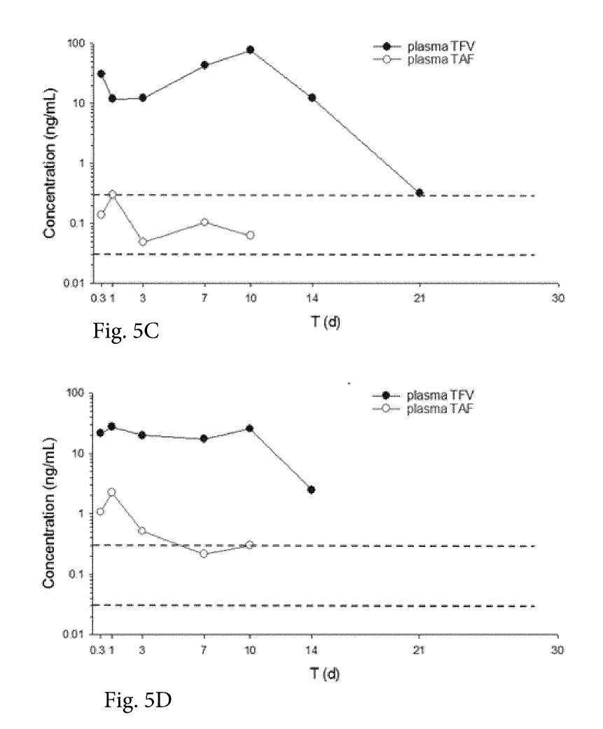

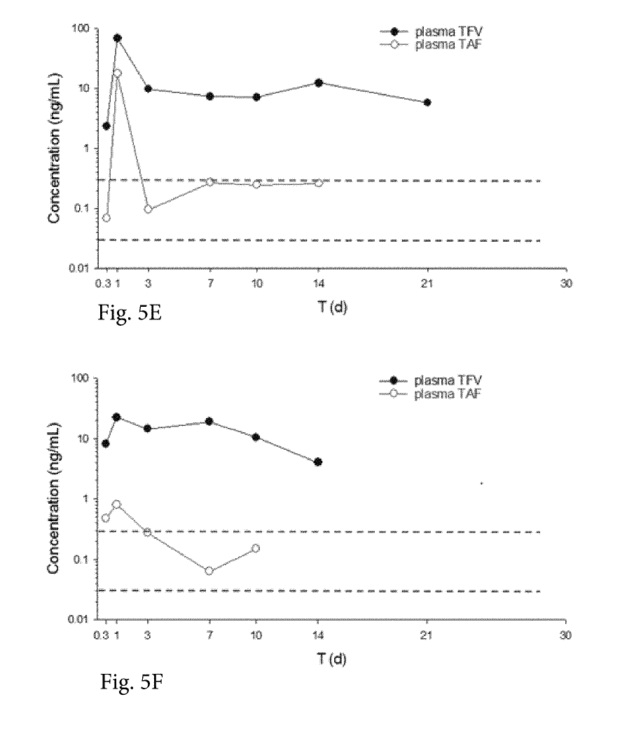

Device 10 can be designed for controlled release of a wide range of therapeutic and preventive active pharmaceutical ingredients (i.e., active agents). Unlike other sustained release technologies, membrane-controlled devices are functionally tunable to achieve zero-order release kinetics, attaining a flat drug release profile and a tight concentration range over several weeks to months. By engineering the porosity to average 1 to 2 times the molecular diameter of a target drug, release rates can be controlled throughout the device lifetime, and an isolated reservoir can provide the necessary therapeutic or preventative payload. This ability to load and protect the drug is critical for therapies that often undergo rapid degradation and clearance. The device design proposed herein will mitigate many of the challenges of sustained delivery. The general mechanism of the proposed device is shown in FIG. 1, wherein films 16 and/or 18 encapsulate reservoir 14 of formulated active pharmaceutical ingredient (API) (i.e., active agent 12). After the device is implanted into a subject as shown in FIG. 2A-2B, passage of biological fluid into the implant solubilizes the active agent (e.g., drug), whereupon the active agent is controllably released from the device via release kinetics dictated by the porous properties of membranes 16 and/or 18 (see, e.g., FIGS. 3-5). FIG. 3 is a line graph showing linear in-vitro release (mg) of TAF from several Thin Film Polymer Devices (TFPDs) of the presently disclosed subject matter over time. Each line represents a single device (TFPD). FIG. 4 shows the same data that is presented in FIG. 3 except that this graph shows the daily release (mg/day) of TAF from the TFPDs. The design of this experiment is detailed in Example 1. Example 2 describes in-vivo studies of New Zealand White rabbits that were subcutaneously implanted with TFPD containing the TAF (or control devices) for 30 days of drug release. FIGS. 5A-5F are graphs of plasma TAF and tenofovir (TFV) concentrations over the course of 30 days from an individual New Zealand White rabbit after subcutaneous implantation with a TFPD containing a high dose target of TAF of 1.5 mg/day.

Thus, the presently disclosed subject matter includes a method for sustained delivery of active agent 12 to a subject, comprising implanting the disclosed reservoir device subcutaneously in a body of a subject. Diffusion of the active agent through the pores of membranes 16 and/or 18 provide sustained delivery of the active agent to the subject for one or a combination of a prevention, treatment, or contraception. In some embodiments, the diffusion of the active agent through the pores of membranes 16, 18 is zero order kinetics at a steady state. In some embodiments, the sustained delivery is a period ranging from 2-3 months. In some embodiments, the prevention is the prevention of infection with HIV.

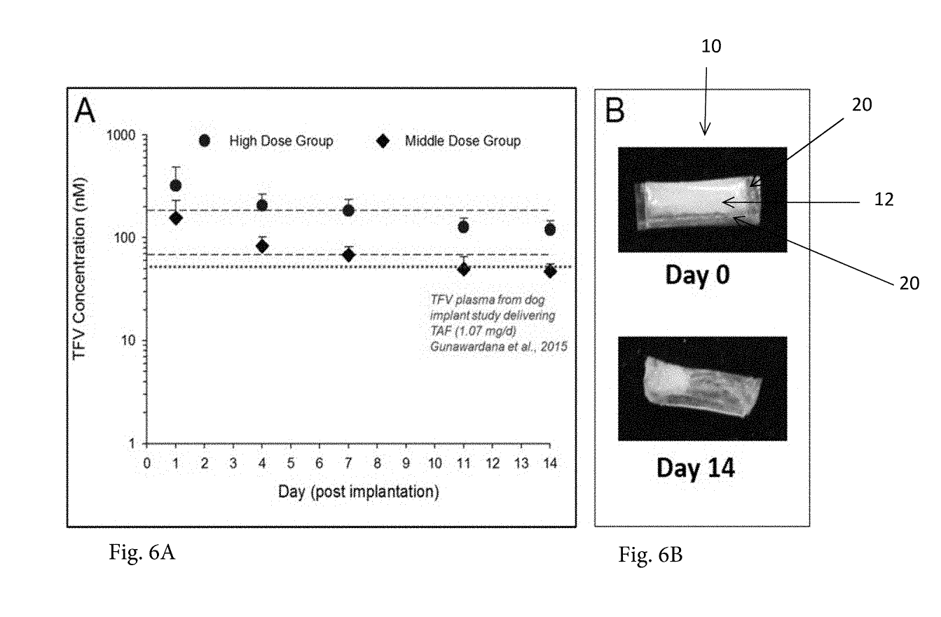

For PrEP specifically, the biostability and required injection volumes of agents constrain delivery technologies. Recent evidence has been published indicating that long-term protection is feasible. However, the persistence is still generally characterized as first order, which requires administration of high dosing and the need for high burden infusion procedures. Device 10 is designed for subcutaneous implantation, which simplifies administration with lower-skilled staff, facilitating access in resource-limited settings. Moreover, this biodegradable product alleviates the need for an extra clinic visit to remove the implant after depletion. Importantly, as shown in FIGS. 6A and 6B, because this technology delivers active drug through a device rather than a gel or nanosuspension, it is reversible and retrievable throughout the duration of treatment. This is beneficial in clinical situations requiring swift removal (e.g., product-related serious adverse event). Additionally, this technology can be developed to simultaneously deliver combinations of biologics, such as antibodies, or small molecules, such as ARV drugs for protection against multiple HIV strains and/or have different mechanisms of action (MOA).

Long-term storage stability presents challenges, as biologics and some small molecule therapeutics inherently have limited stability in the aqueous state where they are subject to chemical and physical degradation, as well as aggregation. By contrast, when lyophilized, the shelf-life of antibodies can be extended, even with room temperature storage in many cases. The presently disclosed devices can utilize API in a dry lyophilized form, packaged within the device and subsequently resolubilized in vivo for release into the subcutaneous space. Alternatively, the API drug can exist as a slurry. Rehydration and release are controlled via the engineered membrane.

Because polymer properties and drug formulations (e.g., connectivity and pore size) affect the release rate of APIs through PCL films, proper design of architectures is crucial to achieve zero-order release kinetics. To this end, the present disclose provides methods to fabricate biocompatible thin PCL films of different properties, including differences in molecular weight, porosities, and films thickness, ultimately tuning release kinetics according to required duration.

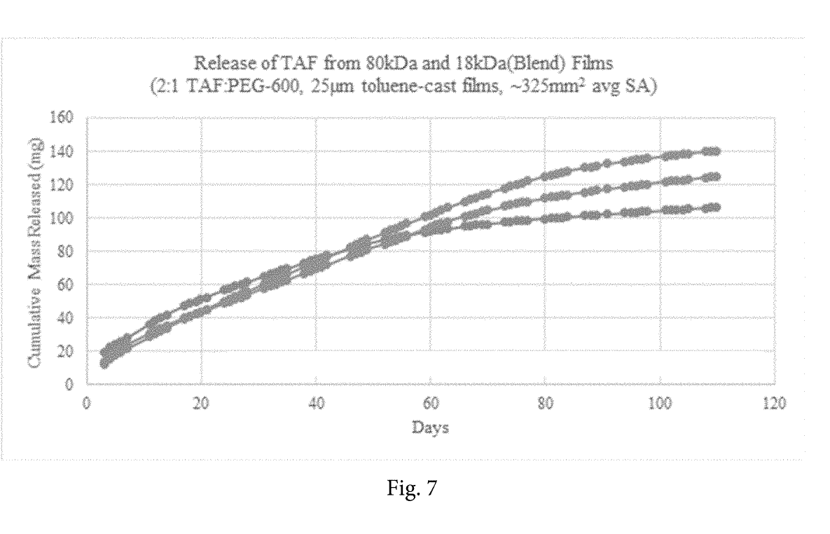

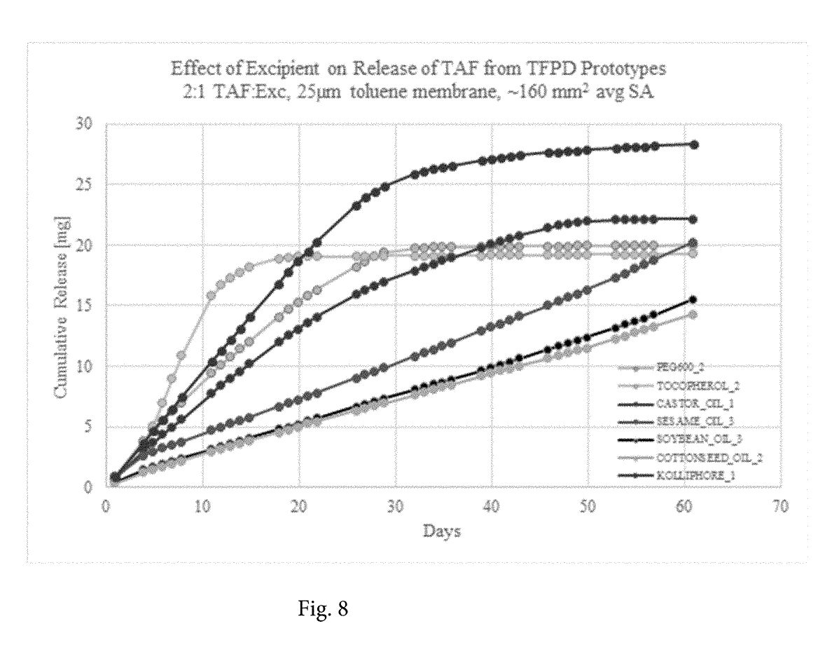

Methods are provided herein in the EXAMPLES for manufacturing and evaluating devices comprising PCL thin films that meet mechanical properties required for device insertion and utilization (FIGS. 2-14). The dimensions and geometry of the devices have been tuned to accommodate injector systems, such as trochar used for the IMPLANON contraceptive implant for hormonal therapy (FIG. 2A-2B). In one example, a PCL thin film device, with an approximate membrane thickness of 25 μm and dimensions of 40×2.5 mm, is capable of holding sufficient antibody for 60 days at a predicted release rate of 2 mg/day (if formulated at a 1:1 weight ratio of antibody:excipient). In another example, with an approximate membrane thickness of 25 μm and dimensions of 40×2.5 mm, the device can hold sufficient Tenofovir Alafenamide Fumarate (TAF), a nucleotide reverse transcriptase inhibitor (NRTI), for 90 days at a predicted release rate of 1 mg/day. Evaluations of similar devices for subcutaneous implantation and sustained delivery of active agents in female New Zealand rabbits (N=35) show that implants remain structurally intact after subcutaneous placement, remaining in-place over 45 days, as designed with an 80 kDa MW PCL film. Other in vivo results show that devices fabricated with 20/80 80 kDa:10 kD PCL porous polymers shows signs of degradation at 2 months. The parameters can be tuned for optimal degradation profiles needed for zero-order release. FIG. 7 shows a graph illustrating the cumulative mass of TAF released (mg) versus time for reservoir devices of 18 kDa blend films (top and middle lines) or 80 kDa blend film (bottom line) according to one or more embodiments of the presently disclosed subject matter. FIG. 8 is a graph illustrating the cumulative release of TAF (mg) from TFPDs having differing excipients over time.

The invention differs from prior devices as follows:

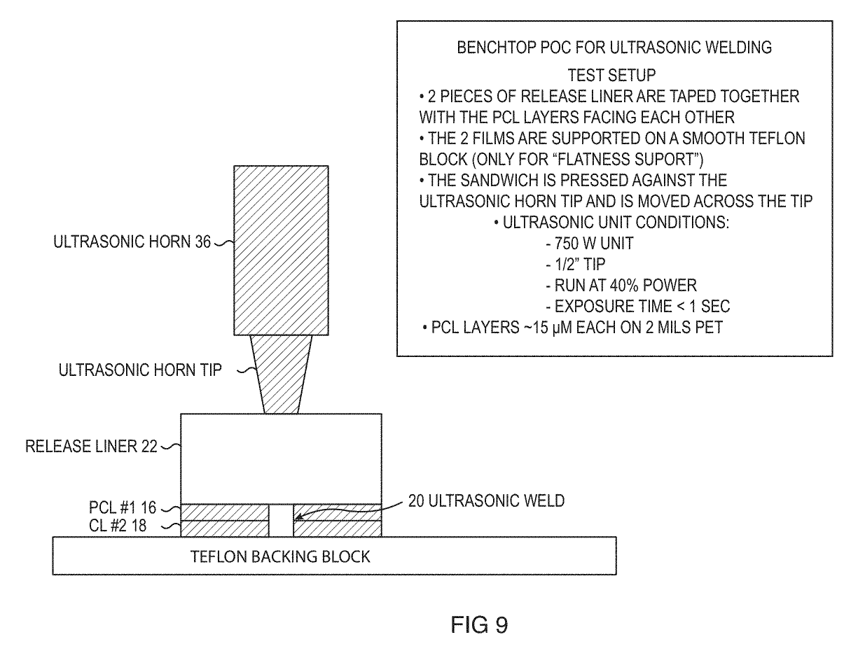

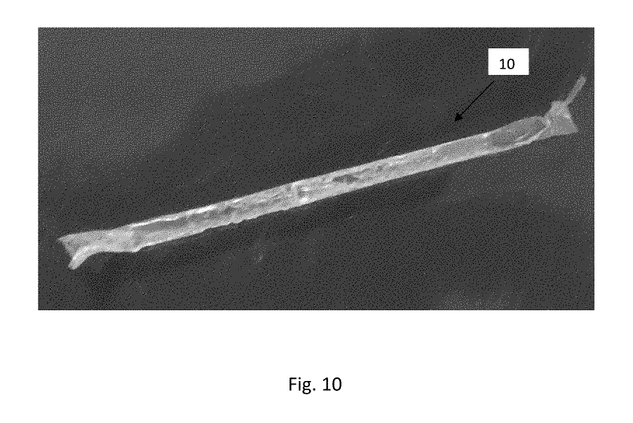

Device 10 is a flexible, permeable polymer film cylinder filled with active ingredient 12 (FIG. 6B and FIG. 10). Qualification tests were performed with the set up diagrams shown in FIG. 9.

In some embodiments, device 10 can be manufactured by folding porous membrane 16 over to define tubular cavity, depositing active agent 12 into the cavity, and applying an ultrasonic force to the porous membrane to create welded seal 20 that contains the active agent within the tubular reservoir 14. Porous membrane 16 allows for diffusion of active agent 12 through the pores of the membrane when device 10 is positioned subcutaneously in a body of a subject.

Alternatively, as shown in FIGS. 9-14, device 10 can be manufactured by imparting vacuum 34 to first porous membrane 16 positioned on mold 30 defining at least one cavity 32, where the first porous membrane takes a shape of the cavity in the presence of the vacuum. Active agent 12 can then be deposited into a portion of the first porous membrane that is received in the cavity. Second porous membrane 18 carried on release liner 22 can be positioned over active agent 12 and in contact with first porous membrane 16. Ultrasonic force 36 can then be applied to release liner 22 positioned over the porous membrane(s) in an area surrounding the active agent to create welded seal 20 to contain the active agent within the cavity to create reservoir 14. Welded membranes 16, 18 can then be released from mold 30 and release liner 22 to provide a reservoir device, where the porous membranes allow for diffusion of active agent 12 through the pores of the membranes when device 10 is positioned subcutaneously in a body of a subject.

In some embodiments, the disclosed method further comprises a distribution of apertures within mold 30 of the cavity 32 to spread vacuum 34 over a broader surface area of the portion of first porous membrane 16 that is received in cavity. In some embodiments, the method further comprises cutting membranes 16, 18 to singulate the reservoir devices.

One or both of first and second porous polymer membranes 16, 18 can comprise polycaprolactone (PCL), poly(lactic-co-glycolic acid) (PLGA), or polylactic acid (PLA). For example, in some embodiments, the first and the second porous polymer membranes comprise polycaprolactone (PCL) at a molecular weight ranging from 15,000-80,000 kDa. Membranes 16, 18 can have a thickness ranging from 1-30 μm or from 10-25 μm.

The use of other thermal processes to achieve fabrication of device 10 (contact heating via conduction only, heated convection air) are not sufficiently controllable and result in damage to the film and failure of the seal. This is due to the sharp melting temperature of PCL and lack of cohesive strength of the melted film.

One skilled in the art will readily appreciate that the present invention is well adapted to carry out the objects and obtain the ends and advantages mentioned, as well as those inherent therein. The present examples along with the methods described herein are presently representative of preferred embodiments, are exemplary, and are not intended as limitations on the scope of the invention. Changes therein and other uses will occur to those skilled in the art which are encompassed within the spirit of the invention as defined by the scope of the claims.

### EXAMPLES

### Example 1

### 45 Day, In-Vitro Release of TAF from a TFPD

In one example, PCL films were fabricated by a solution casting method. 80 kDa PCL was dissolved at 7.0% wt in toluene and cast onto a glass substrate using a 25 mil slot to achieve a dry film thickness of 25 μm. Thickness was measured with a micrometer. The Tenofovir Alafenamide Fumarate (TAF), a nucleotide reverse transcriptase inhibitor (NRTI), formulation was prepared by combining TAF and PEG₆₀₀ at a 2:1 mass ratio and hand stirred until combined with a stainless-steel spatula. PCL films were then formed into tubes of the appropriate diameter and long welds were formed along the length of the tube using an impulse heat sealer. Formulated TAF was loaded into the tube by hand using a plastic funnel and plastic rod to within ±1 mg of the target mass and recorded. Drug was subsequently packed tightly into the tube with the plastic rod and seals were made using the impulse sealer at the target device length. After characterization devices were packaged into amber glass vials, labeled serially and shipped to Steris Isomedix Services for terminal sterilization by gamma irradiation at a standard dose of 25 kGy (“Gamma Engineering Run” service was used which does not include sterility validation).

For the in-vitro studies, TFPD implants were submerged in 30 mL of phosphate buffered saline (1X PBS, pH=7.2) in conical centrifuge tubes and kept in a 37° C. shaking incubator at 120 rpm. At a frequency of approximately 48-72 hours samples of buffer were quantified for TAF mass and devices transferred to new buffer solution. Quantification of TAF was performed by ultraviolet-visible absorbance at 260 nm, measured using a spectrophotometer for a 200 μL sample volume in a 96-well quartz microplate. UV absorbance at 260 nm was correlated to TAF concentration in 1X PBS buffer for a range of concentrations between 0.003-0.250 mg/mL. For samples above the absorbance maximum of the instrument, samples were diluted by a factor of 10 and measured again (1000 μL sample diluted in 900 μL of 1×PBS).

Results show the linear release of TAF (in-vitro) from the TFPDs over time (FIG. 3). Four devices (i.e., TIP-PC-004, #7, #8, #9, #12) released with a zero order kinetic profile over time, with an average release rate of 1.4±0.1 mg/day for 45 days. In this case, the devices were prepared using a heat seal, and therefore showed some variance. For example, two devices (i.e., TIP-PC-004-#11, #10) deviated from the predicted release rates at 2.4 mg/day and 1.0 mg/day of TAF, respectively. The same data is presented in FIG. 4, except this graph shows the daily release rates for TAF.

### Example 2

### 30 Day, In-Vivo Release of TAF from a TFPD

For the in-vivo studies, twenty-one female New Zealand White rabbits were subcutaneously implanted with TFPD containing the TAF (or control devices) for 30 days of drug release. In an effort to investigate release tailing at the end of the device lifetime (i.e., drug depletion at Day 30), half of the animals continued for an additional 15 days and were euthanized on Day 45. Three dose groups were used: high dose (HD) targeted at 1.5 mg/day, medium dose (MD) targeted at 0.8 mg/day, and low dose (LD) targeted at 0.15 mg/day.

Blood samples for plasma TAF, TFV and PBMC TFV-DP were collected at predetermined intervals up to 45 days. After blood collection by cardiac puncture under pentobarbital sodium anesthesia, the animals were euthanized via an overdose of intravenous pentobarbital sodium. Blood for drug content were collected into tubes containing K₃ EDTA and then centrifuged to obtain plasma before freezing, and stored at −80° C. Target tissues (vagina, cervix, rectal) and tissues surrounding the implant were placed in cryovials, snapped frozen in liquid nitrogen before freezing and stored at −80° C. Tissues (near implantation) for histopathology were collected into tubes containing formalin phosphate for 48 hours, transferred to another tube containing cold phosphate buffered saline before refrigeration and stored at −4° C. Tissues (near implantation) for inflammatory markers were placed in cryovials, snapped frozen in liquid nitrogen before freezing and stored at −80° C.

FIG. 5 shows an example of in vivo release of TAF from TFPDs. The graphs show individual plasma concentrations of TAF and TFV resulting from delivery of TAF from a TFPD with a HD target of 1.5 mg/day.

### Example 3

### 15 Day, In-Vivo Release of TAF from a TFPD

In vivo implant studies: To assess the in vivo behavior of the implantable devices for PrEP, preliminary studies were conducted in female Sprague-Dawley rats (N=35) over 14 days. Devices fabricated using poly(caprolactone) (PCL) and containing a TAF/PEG₃₀₀ formulation were subcutaneously implanted into the dorsal of the neck of the rats via standard microsurgical techniques. By tuning the device surface area, devices were designed for two different release profiles: high TAF dose (1.1 mg/d) or middle TAF dose (0.45 mg/d) (FIG. 6A). Subsequent to a slight burst of TAF on d=1, the TFV concentration in plasma maintained fairly steady levels over the 14-day period, maintaining TFV plasma levels at or above those reported from a recent TAF implant study in dog (Gunawardana, Remedios-Chan et al. 2015), which relates to predicted TFV levels conferring 92% protection in the recent iPRex clinical study (Grant, Lama et al. 2010). In this rat study, the devices were purposely designed to remain intact over 14 days to enable removal upon TAF depletion. Retrieved devices were stable and intact without observable biofilm formation at d=14 (FIG. 6B); post-mortem examinations showed minimal adverse signs of tolerability with respect to inflammation or morphological abnormalities at the implantation site.

### Example 4

### Characterization of PCL Films

PCL films can be tuned to meet the requisite biodegradation properties (i.e., optimize the time between depletion of API and film biodegradation). For example, 80 kDa MW PCL films exhibit an extended rate of biodegradation, typically on the order of >24 months. Table 1 shows a variety of new PCL film formulations considered. The PCL blended formulations must be tuned for two properties: adequate rates of biodegradation in relation to API release and mechanical robustness. The balance between these two factors proves critical for these thin films. For example, although PCL films comprising 10 kDa demonstrate faster biodegradation than 80 kDa films, the 10 kDa films failed to remain mechanically intact after casting and crumbled upon handling.

TABLE 1
PCL film formulations.

| | | | | | “Fragmentation” | “Dissolution” |
|---|---|---|---|---|---|---|
| | Mass Ratio | | | Average M_{N} | Months until | Months until |
| | 10 kDa | 45 kDa | 80 kDa | (kDa) | M_{N} = 13 kDa* | M_{N} = 5 kDa* |
| A | | | 1 | 80 | 20 | 30 |
| B | | 1 | 1 | 57.6 | 16 | 27 |
| C | | 1 | | 45.0 | 14 | 24 |
| D | 1 | | 8 | 45.0 | 14 | 24 |
| E | 1 | | 1 | 17.8 | 3 | 14 |
| F | 2 | | 1 | 14.1 | 1 | 11 |
| G | 3 | | 1 | 12.8 | — | 10 |

A 17.8 kDa average M_{N} blend (Formulation E in Table 1 above) performed well in release evaluation with both caffeine and TAF. A 90 day evaluation was completed using 2:1 TAF:PEG₆₀₀ in 80 kDa (standard) and 17.8 kDa (blend, formulation E above) PCL devices to (1) demonstrate 90 day sustained release and (2) evaluate any effects biodegradation may have on release. Of the six devices made, three (80k_1, 80 k_2, and 17k_1) demonstrated non-linear release indicative of fabrication defects and their data are not presented. The remaining three devices which performed as designed (80k_3, 17 k_2 and 17k_3) are presented in FIG. 7. Release linearity was maintained through 60 days with rates averaging 1.4+/−0.2 mg/day and device core depletion occurred between 60 and 90 days. Although 90 days of sustained release is possible from a 2.5×40 mm device at a 1 mg/day release rate, with rates of 1.4 mg/day it was not possible to load enough TAF to sustain release for that duration in this study.

### Example 5

### Excipients for TAF

Excipients on the FDA GRAS list of acceptable compounds were evaluated for TAF stability and short-duration TAF release. Devices containing TAF in combination with selected GRAS excipients were evaluated in prototype devices 60 days, presented in FIG. 8. Surface area normalized release rates (normalized to 314 mm²) ranged between 0.4 and 3.5 mg/day are summarized in Table 2. Of note, soybean oil and sesame oil were not selected, as these two excipients showed slightly higher variability, as compared to cottonseed oil. Tocopherol was not chosen because of the high release rate of TAF near 3.5 mg/day.

TABLE 2
Summary of screened excipient stability and TAF release data

| | TAF | Chromatographic | Release |
|---|---|---|---|
| | Solubility¹ | Purity¹ | Rate^{2,3} |
| Excipient | (mg/mL) | (%) | (mg/day) |
| Kolliphor EL | 15.3 | 98.78 | 1.71 ± 0.39 |
| (PEG-35 castor oil) | | | |
| Tocopherol | 10.1 | ND | 3.56 ± 1.25 |
| (Vitamin E) | | | |
| Soybean oil | 0.105 | ND | 0.44 ± 0.15 |
| Cottonseed oil | 0.207 | ND | 0.50 ± 0.02 |
| Sesame oil | 0.095 | ND | 0.52 ± 0.2 |
| Castor Oil | 6.7 | 99.83 | 1.40 ± 0.02 |
| PEG 600 | 5.4 | 95.95 | 1.87 ± 0.26 |
| TAF std. | — | 99.88 | — |

ND = None Detected
¹Excess TAF dissolved in excipient at 37° C., quantified by HPLC
²Slope of cumulative release through 15 days normalized to 314 mm²
³25 μm 80 kDa PCL film

### Example 6

### Manufacturing of Medical Devices

Specific information regarding ultrasonic welding for medical device manufacture:

Qualification tests were performed with the set up diagrams provided in FIG. 9.

An example of the fabricated reservoir device structure is represented in FIG. 10.

The structure was created in accordance with the following procedure.

Coating Solution Preparation:

The PCL film layer was produced using a 16% by weight solution of SIGMA ALRDRICH Polycaprolactone (average MW 45,000) dissolved in toluene. The solution is prepared by weighing the appropriate amounts of material into a sealable glass container and allowing the polymer to dissolve over the course of several days at room temperature. The mix is initially agitated with a vortex unit; however, the polymer forms gels which do not mix easily. In addition to toluene, acetone and other solvents may be used. If these solvents are used, it is necessary to carry out the dissolution process at a slightly elevated temperature (˜30 C). The expected concentration range allowable for roll-to-roll production of the film is 5% to 20% by weight. Other polymer molecular weights can be viable (eg. PCL 15,000-80,000) and other polyester films, or copolymers with PCL can be used (e.g. PLGA, PLA).

Coating Process:

Film samples are produced in a bench top coating system using a fixed gap set up and a smooth stainless steel rod. The gap is set to 10 mils to produce a liquid film ˜10 mils (250 microns). To carry out the coating, a disposable pipette is used to dispense fluid onto a silicone release film (carrier web), (Flexmark 200 Poly Sc-6 Liner). The dispensed liquid is drawn across the carrier web to create a uniform liquid layer. The coated liquid is dried with a hand held convection dryer set at 300 F and positioned ˜10 inches above the liquid. The coated liquid is dried until the layer has a “uniform” hazy appearance. Additional drying results in melting of the PCL layer which is seen by the layer becoming “clear” (indicating that the polymer temperature is >58 C. Removal of the heat gun after melting results in the film returning to having a hazy appearance. The hazy appearance occurs initially when the coated layer temperature reaches ˜30 C (measured with IR Thermometer).

Other coating processes expected to be usable include slot die, cascade, gravure, reverse roll in a r2r fabrication line. The drying process on such a coating line could include a drying zone, as well as a post-dry baking zone which melts the PCL and allows reduction of the residual solvent level in the finally dried PCL. Such a temperature condition could be held to approximately 60 C to limit deformation of the carrier web.

The carrier web can be a variety of release films having a range for the thickness. The preferred thickness is 2 mils to limit the waste after carrier web use. Films, thinner than 2 mils (eg. 1 mil commercially available films), have mechanical properties which are more susceptible to wrinkling in a process, and may be more sensitive to shrinkage.

Final dried film thickness can be adjusted by solids concentration in the coating fluid and by the wet thickness coated. This range can be adjusted from 1 micron up to 30 microns. The preferred thickness is 20 microns. 10 microns is probably a lower limit for use in the device fabrication process described below. A film thickness greater than 20 microns may limit the efficacy of the drug release properties.

The above coating process could also be carried out with the carrier web being in a sheet format, rather than r2r. In this case, gravure and reverse roll application processes would not be used.

Measurement:

Film thickness is measured using a Mitutoyo “snap gauge” No. 28049-10.

Device Fabrication:

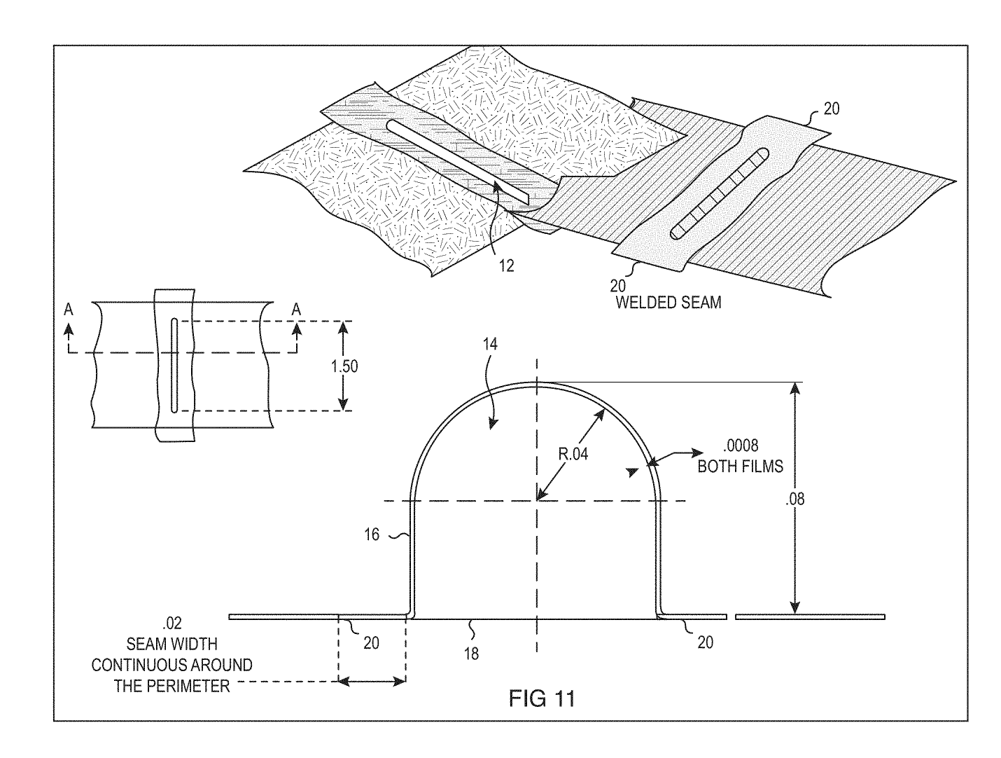

A layout of the device fabrication is shown in FIG. 11.

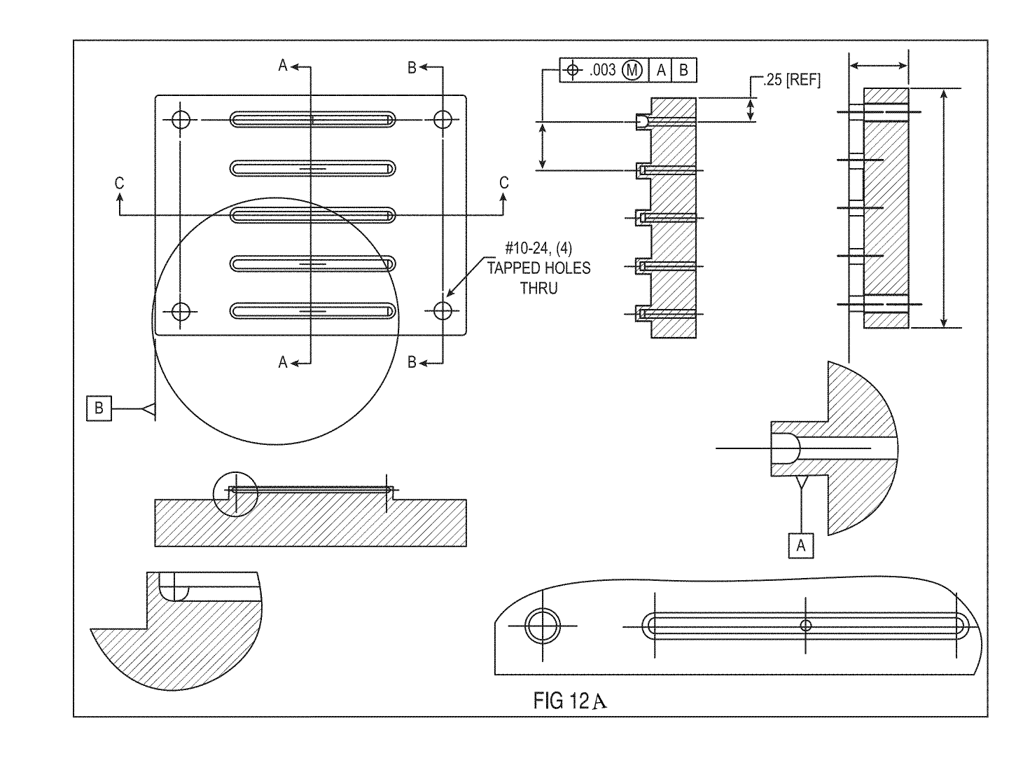

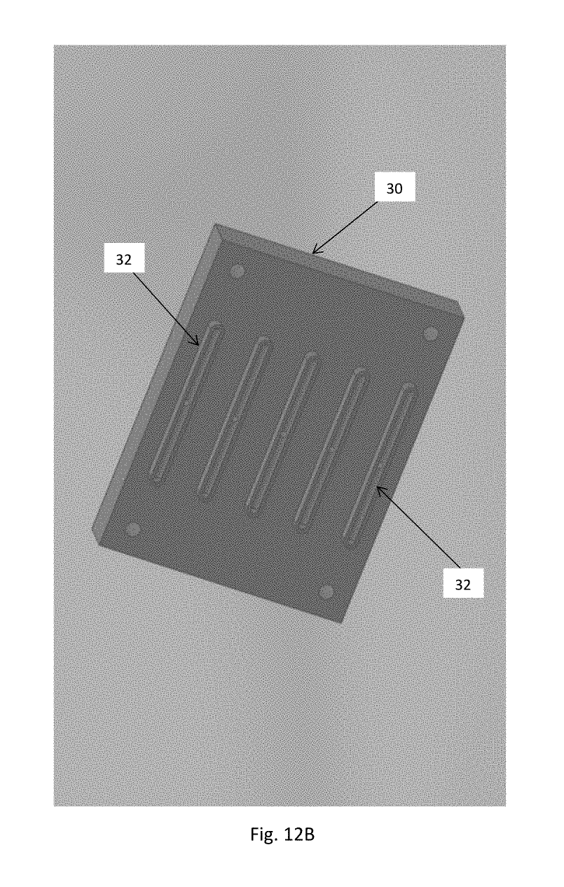

The dried PCL film is removed from the carrier web and place across the cavity shaping die in FIGS. 12A and 12B.

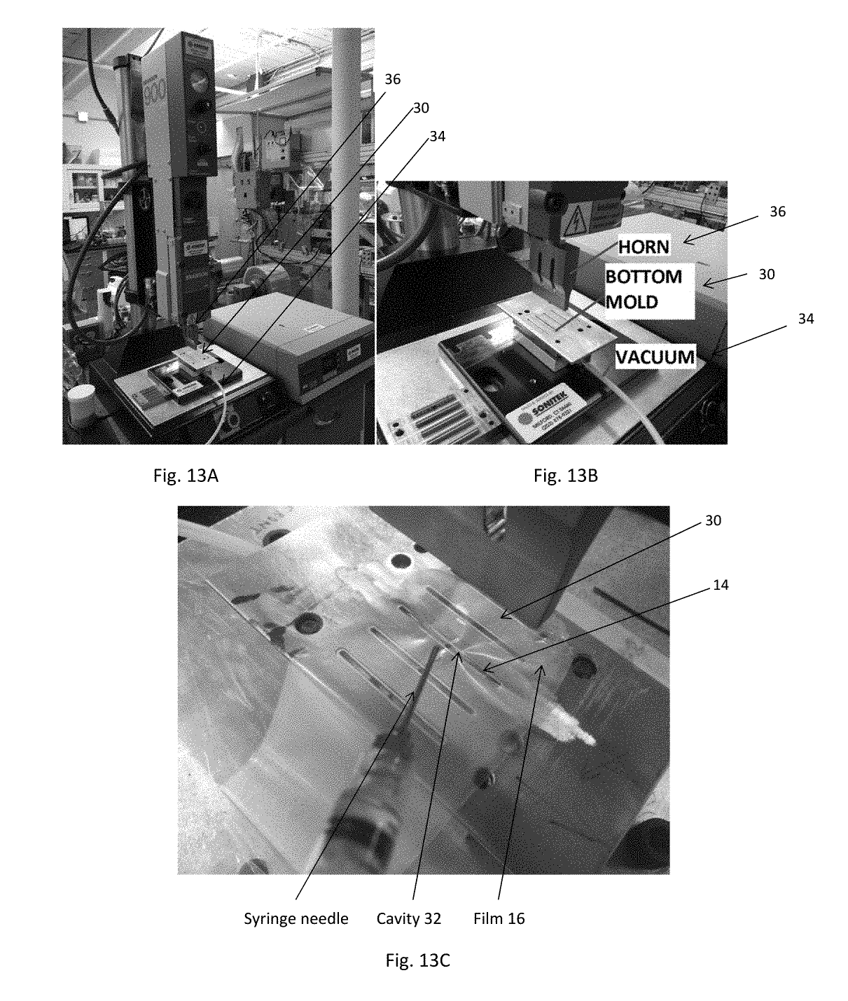

The ultrasonic welding process utilizes the Branson model 941AE/947DA ultrasonic welding system and custom fixtures for forming the cylindrical cavity, filling the cavity, and final sealing as shown in FIGS. 13A-13C.

To fabricate a device,
The first PCL layer is removed from the release liner substrate and is placed manually onto the bottom mold. The vacuum pump is turned on. Application of vacuum forces the PCL layer to form a recess by taking the shape of the slot in the mold under the vacuum.
A syringe is used to fill the recess with the gel (glycerin) (FIG. 13A-13C). The gel was used as a demonstration material but did not contain an active drug.
The second layer of the PCL, still attached to the release liner is positioned on the top of the first PCL layer (with the filled cavity).
The release liner is on top (having the two PCL layers in contact with one another).
The welding process is initiated and the horn pneumatically moves down in contact with the top surface of the release liner. This step engages the two PCL films under a certain pressure to produce an ultrasonically welded seam.
The final filled device is removed from the release liner easily.

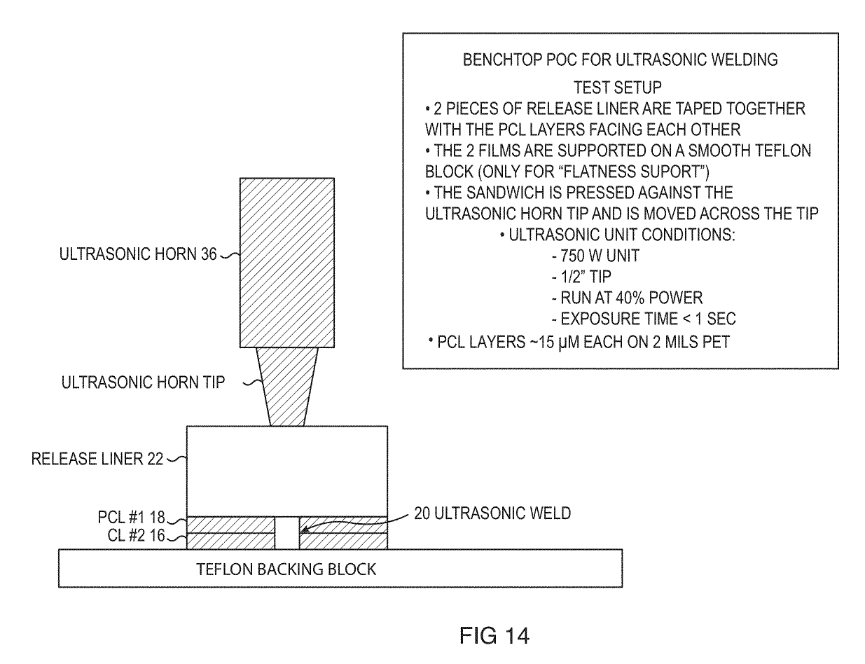

Repeating the drawing shown in FIG. 9, a general layout of the various webs and layers is shown in FIG. 14, without the cavity formation on PCL#2. The inclusion of the carrier web for PCL#1 is necessary to eliminate “stickage” of the PCL to the ι₃₄ asonic horn surface. After welding is completed, the coupled PCL films can be removed from both the Teflon backing block surface and the carrier web for PCL#1.

Important aspects of the process:
Ultrasonic welding in a precise location of the perimeter of the cylindrical shape
Controlled vacuum to form the PCL cylinder in the cavity without damaging the PCL film
A distribution of vacuum holes inside the cavity to spread the vacuum source over a broader surface area
A flat surface (land area) around the cavity to allow the film to be tensioned uniformly across the cavity, allowing film shaping into the cavity via vacuum
Teflon (or other not stick coatings) on the cavity and flat land area to prevent the PCL from sticking to these surfaces
Drug carrier (matrix) can be a liquid, gel, paste, powder, or other form. The technique and tools used to fill the cavity with the material will be dependent on the form of the filler.

Any patents or publications mentioned in this specification are indicative of the levels of those skilled in the art to which the invention pertains. These patents and publications are herein incorporated by reference to the same extent as if each individual publication was specifically and individually indicated to be incorporated by reference.

1. A reservoir device comprising an active agent contained within a reservoir, the reservoir defined by one or more porous polymer membranes sealed with an ultrasonic weld, the porous membrane allowing for diffusion of the active agent through the pores of the membrane when positioned subcutaneously in a body of a subject.

2. The device of claim 1, wherein the porous polymer membrane comprises polycaprolactone (PCL), poly(lactic-co-glycolic acid) (PLGA), or polylactic acid (PLA).

3. The device of claim 1, wherein the porous polymer membrane comprises polycaprolactone (PCL) at a molecular weight ranging from 15,000-80,000 kDa.

4. The device of claim 1, wherein the porous polymer membrane has a membrane thickness ranging from 1-30 μm or from 10-25 μm.

5. The device of claim 1, wherein the active agent is one or a combination of a therapeutic, a preventative, or a contraceptive.

6. The device of claim 5, wherein the active agent comprises an antibody, a small molecule, a protein, or a peptide.

7. The device of claim 1, wherein a size of the pores of the porous polymer membrane ranges from 1-2 times the diameter of the active agent.

8. The device of claim 1, wherein the active agent comprises an antibody for the prevention of HIV infection.

9. (canceled)

10. The device of claim 1, further comprising an excipient contained within the reservoir.

11. (canceled)

12. The device of claim 1, wherein the porous polymer membrane is biodegradable.

13. The device of claim 1, wherein the reservoir has a cylindrical shape.

14. (canceled)

15. A method for manufacturing a reservoir device for delivery of an active agent to a subject, the method comprising:
imparting a vacuum to a first porous membrane positioned on a mold defining at least one cavity, wherein the first porous membrane takes a shape of the cavity in the presence of the vacuum;
depositing an active agent into a portion of the first porous membrane that is received in the cavity;
positioning a second porous membrane carried on a release liner over the active agent and in contact with the first porous membrane;
applying an ultrasonic force to a release liner positioned over the porous membrane(s) in an area surrounding the active agent to create a welded seal to contain the active agent within the cavity; and
releasing the welded porous membranes from the mold and the release liner to provide a reservoir device(s), the porous membranes allowing for diffusion of the active agent through the pores of the membrane when the reservoir device is positioned subcutaneously in a body of a subject.

16. The method of claim 15, further comprising a distribution of apertures within the mold of the cavity to spread the vacuum over a broader surface area of the portion of the first porous membrane that is received in the cavity.

17. The method of claim 15, further comprising cutting the porous membranes to singulate the reservoir devices.

18. The method of claim 15, wherein one or both of the first and the second porous polymer membranes comprise polycaprolactone (PCL), poly(lactic-co-glycolic acid) (PLGA), or polylactic acid (PLA).

19. The method of claim 15, wherein the first and the second porous polymer membranes comprise polycaprolactone (PCL) at a molecular weight ranging from 15,000-80,000 kDa.

20. The method of claim 15, wherein the first and the second porous polymer membranes have a membrane thickness ranging from 1-30 μm or from 10-25 μm.

21. (canceled)

22. (canceled)

23. (canceled)

24. (canceled)

25. (canceled)

26. (canceled)

27. (canceled)

28. (canceled)

29. (canceled)

30. (canceled)

31. A method for sustained delivery of an active agent to a subject, the method comprising implanting the reservoir device of claim 1 subcutaneously in a body of a subject, wherein diffusion of the active agent through the pores of the membrane of the device provides sustained delivery of the active agent to the subject for one or a combination of prevention, treatment, or contraception.

32. The method of claim 31, wherein the prevention is prevention of infection with HIV.

33. (canceled)

34. (canceled)

35. The method of claim 31, wherein the reservoir device is biodegradable.

36. (canceled)

37. (canceled)

38. (canceled)
